(19) United States
(12) Patent Application Publication
(10) Pub. No.: US 2020/0397628 A1
(43) Pub. Date: Dec. 24, 2020

(54) THREE-DIMENSIONAL SHEET MATERIAL AND ABSORBENT ARTICLES INCLUDING SUCH MATERIAL

(71) Applicant: KIMBERLY-CLARK WORLDWIDE, INC., Neenah, WI (US)

(72) Inventors: Jina Gwag, Yongin (KR); HyungWoo Park, Suwon-si (KR); JuHyung Lee, Yongin-si (KR); SeongDae Roh, Yongin-si (KR)

(21) Appl. No.: 16/088,146

(22) PCT Filed: Mar. 31, 2016
(86) PCT No.: PCT/US16/25157

§ 371 Date: Sep. 25, 2018

(51) Int. Cl. A61F 13/511; B32B 3/26; B32B 3/28; B32B 5/02; B32B 7/12; B32B 7/09; A61F 13/47; A61F 13/512

(52) U.S. Cl. CPC A61F 13/51104; B32B 3/266; B32B 3/28; B32B 5/022; B32B 7/12; B32B 2555/02; A61F 13/47; A61F 13/5122; B32B 2307/726; B32B 2262/02; B32B 7/09

(57) ABSTRACT

A three-dimensional sheet material can be provided with a pattern of various material treatments. The material treatments can include a pattern of embossments and a pattern of apertures. The three-dimensional sheet material can be utilized in a personal care absorbent article including, but not limited to, diapers, training pants, incontinence products, feminine hygiene products such as sanitary napkins and panty liners, as well as other absorbent article such as bandages and wound dressings.

### BACKGROUND OF THE DISCLOSURE

Absorbent articles such as diapers, training pants, incontinence products, feminine hygiene products, as well as health care related products such as bandages and other wound dressings have a common goal of rapidly absorbing discharged body fluids such as blood, menses, urine and bowel movements. Typically such absorbent articles will have a body contacting side and surface which is near or in contact with the wearer's skin, some type of absorbent core and a backsheet layer that will prevent the retained fluid from exiting the absorbent article and possible soiling the surrounding areas including the wearer's clothes.

Thus, it is desirable for such absorbent articles to rapidly take in discharged body fluids, pass them to subadjacent layers in the absorbent article and provide air circulation adjacent the wearer's skin to promote skin wellness. Air circulation allows drying of the skin to prevent skin irritation such as diaper rash in the case of diapers, training pants, and incontinence absorbent articles. Air circulation also provides increased comfort by allowing the body contacting material, often referred to as a topsheet layer, to dry out. In addition, comfort and dryness can be further enhanced by minimizing the amount of the topsheet layer that is in direct contact with the skin. This also facilitates a reduction in what is called “rewet” which is the backflow of fluid from the absorbent core into the topsheet layer.

While many absorbent articles have attempted to provide topsheet layers with the desired body exudate handling properties, there is still a need for an improved material in this regard. There is a need for a three-dimensional material which can be used in this regard in conjunction with personal care absorbent articles including, but not limited to, diapers, training pants, incontinence products, feminine hygiene products such as sanitary napkins and panty liners, as well as other absorbent article such as bandages and wound dressings.

### SUMMARY OF THE DISCLOSURE

In various embodiments, a three-dimensional sheet material can be characterized by comprising a first layer of material comprising an upper surface and a lower surface defining a thickness therebetween; a plurality of first embossments in which each first embossment defines a first major axis and a first minor axis, and a first overall length; a plurality of second embossments in which each second embossment defines a second major axis and a second minor axis, and a second overall length; a plurality of first apertures in which each first aperture has a first width and a sidewall defining a first depth; a plurality of second apertures in which each second aperture has a second width and a sidewall defining a second depth; wherein the first major axis is perpendicular to the second major axis +/−10 degrees; the first width of at least a portion of the plurality of first apertures is less than the second width of at least a portion of the plurality of the second apertures; and the first depth of at least a portion of the plurality of first apertures is less than the second depth of at least a portion of the plurality of the second apertures.

In various embodiments, the plurality of first embossments extend upwardly and away from the upper surface; the plurality of second embossments extend upwardly and away from the upper surface; the first embossments have opposed first ends separated by a first central region having opposed first sides, each of the first ends having a first end width and the first central region having a first central width, the first central width being less than the first end width of each of the first ends, and the second embossments have opposed second ends separated by a second central region having opposed second sides, each of the second ends having a second end width and the second central region having a second central width, the second central width being less than the second end width of each of the second ends.

In various embodiments, the plurality of second apertures are adjacent the ends of the plurality of first embossments and the plurality of second apertures are also adjacent the second ends of the plurality of second embossments. In various embodiments, the plurality of first apertures are adjacent the opposed first sides of the first central region of the plurality of first embossments and the plurality of first apertures are also adjacent the opposed second sides of the second central region of the second embossments.

In various embodiments, the second ends of the plurality of second embossments are separated from the first central regions of the plurality of first embossments by the first apertures. In various embodiments, the first ends of the plurality of first embossments are separated from the second central regions of the plurality of second embossments by the first apertures.

In various embodiments, the second depth of the plurality of second apertures is greater than the first depth of the plurality of first apertures as measured from the upper surface.

In various embodiments, a second layer of material is adjacent the lower surface of the first layer of material, the second layer of material having a top surface and a bottom surface separated by a second thickness. In various embodiments, the plurality of 1st apertures and the plurality of second apertures extend through the first layer of material and the second layer of material. In various embodiments, the second layer of material is bonded to the first layer of material.

In various embodiments, a personal care absorbent article can include the three-dimensional sheet material. In various embodiments, the personal care absorbent article can comprise a topsheet layer and a backsheet layer with an absorbent core located between the topsheet layer and the backsheet layer wherein the topsheet layer includes the three-dimensional sheet material. In various embodiments, the absorbent article is a sanitary napkin.

### BRIEF DESCRIPTION OF DRAWINGS

FIG. 1 is a top plan view of an exemplary embodiment of a three-dimensional sheet material.

FIG. 2 is a cross-sectional side view of the three-dimensional sheet material of FIG. 1 taken along line 2-2.

FIG. 3 is an exemplary embodiment of a surface of an embossing roll which can be used in the manufacture of a three-dimensional sheet material.

FIG. 4 is an exemplary embodiment of a surface of an embossing roll which can be used in the manufacture of a three-dimensional sheet material.

FIG. 5 is a top plan view of a three-dimensional sheet material formed by intermeshing embossing rolls.

FIG. 6 is a perspective view of a three-dimensional sheet material with the lower surface facing the viewer.

FIG. 7 is a cut-away top plan view of an exemplary embodiment of an absorbent article, in this case, a feminine hygiene product utilizing the three-dimensional sheet material as a topsheet layer.

Repeat use of reference characters in the present specification and drawings is intended to represent the same or analogous features or elements of the disclosure.

### DETAILED DESCRIPTION OF THE DISCLOSURE

The present disclosure is directed towards a three-dimensional sheet material. The three-dimensional sheet material can be provided with a pattern of various material treatments. The material treatments can include a pattern of embossments and a pattern of apertures. The three-dimensional sheet material can be utilized in a personal care absorbent article including, but not limited to, diapers, training pants, incontinence products, feminine hygiene products such as sanitary napkins and panty liners, as well as other absorbent article such as bandages and wound dressings.

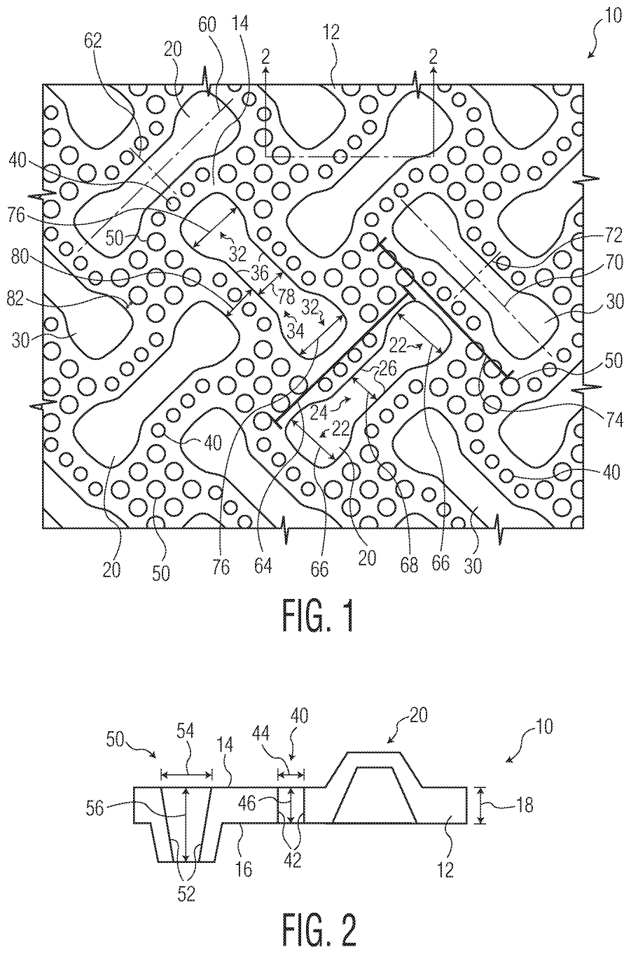

Referring to FIGS. 1 and 2, there is shown in FIG. 1 a top plan view of an exemplary embodiment of a three-dimensional sheet material and, in FIG. 2, a cross-sectional view of the three-dimensional sheet material of FIG. 1 taken along line 2-2. The three-dimensional sheet material 10 has at least a first layer of material 12 which is subjected to embossing and aperturing. The material 12 is any material which is able to pass liquids and gases, also referred to as being fluid permeable. In the context of personal care absorbent articles, the material 12 should be able to readily pass liquids such as urine and/or menses depending the particular end application. Such is the case when the end application is a feminine care product such as a sanitary napkin, pantiliner, or other feminine care absorbent article. In addition, in certain instances it may be desirable for the material 12 to be able to pass portions of more solid-type materials such as runny bowel movements when the end application is for such products as diapers, training pants, and incontinence garments. Further, the material 12 may be used for other applications wherein fluid transfer and absorption is necessary as in the case of bandages and other health care related products.

Due to the fact that the material 12 is subjected to embossing techniques and aperturing techniques to form the three-dimensional structure, the material 12 should be selected from a material that is able to hold a predetermined shape due to the application of pressure and optionally the use of heat. The application of pressure and heat can be achieved by a variety of methods including the use of engraved embossing rolls with optional heat and/or cooling as well as by the use of ultrasonics. In addition, it is desirable that the material be soft to the touch, especially when the material is being used as a topsheet layer of a personal care absorbent article which is designed to be worn against the skin. Due to the need to be set in a three-dimensional shape, in some instances it is also desirable that the material 12 contain at least some material made from a polymeric plastic material and/or other material that will take a pressure and/or heat set. Examples of such materials include, but are not limited to, polyolefins, polyesters, poly vinyl alcohol, polyurethane, nylon and the like. It is also possible to form the material 12 from varying materials, both natural and synthetic such as blends of polymeric fibers and natural fibers such as cotton and wood pulp fluff. Other suitable materials include spunbond materials, meltblown materials, staple fiber bonded carded webs, through air bonded carded webs, chemically bonded carded webs, airlaid webs, spunlace webs, and hydroentangled webs. It is also possible to use multilayer materials such as laminates of layers of spunbond material and meltblown material.

Due to the fact that the materials of the present disclosure can be used for applications where fluid permeability is not critical or the material only has to be gas or vapor permeable, it is also possible to form the material 12 from a plastic film such as a thermoplastic film made from polymeric materials such as polyolefins and other materials that may be embossed using heat and/or pressure or ultrasonics so as to hold a three-dimensional shape.

Still further, it is possible to form the material 12 from other multi-layered structures such as to or more layers of nonwovens or a combination of nonwoven and film layers. Such multi-layer structures may be formed in advance of applying the three-dimensional embossing and aperturing or they may be joined as a result of the three-dimensional embossing and aperturing process

The first layer of material 12 can have an upper surface 14 and a lower surface 16 defining a thickness 18 therebetween. To provide the three-dimensional shape to the sheet material, the material 12 is provided with a plurality of first embossments 20 extending upwardly and away from the upper surface 14. The material 12 also includes a plurality of second embossments 30 extending upwardly and away from the upper surface 14. In addition to having the first embossments 20 and the second embossments 30, the material 12 is provided with a plurality of first apertures 40 which extend between the upper surface 14 and the bottom surface 16 of the material 12. The material 12 further includes a plurality of second apertures 50 which extend downwardly and away from the lower surface 16 of the material 12.

The overall thickness 18 of the three-dimensional sheet material 10 will depend on the particular end application. In the context of personal care absorbent articles, the three-dimensional sheet material 10 can be used as a part of the topsheet layer. The three-dimensional sheet material 10 can also be used as one of the intermediate layers of a personal care absorbent article including the absorbent core or one or more of the other layers of such products including what is commonly termed the surge or transfer layer which is a layer between the absorbent core and the topsheet layer. Furthermore, the three-dimensional sheet material 10 can be used as a layer between the absorbent core and the backsheet layer of the personal care absorbent article. In various embodiments, the thickness 18 of the three-dimensional sheet material 10 can be from about 0.1, 0.3, 0.5, 0.8 or 1 mm to about 1.2, 1.5, 2, 3, or 4 mm.

As to basis weight, again the actual basis weight can vary depending on the end application. In the context of personal care absorbent articles, the three-dimensional sheet material 10 can have a basis weight from about 8, 16, 22, or 35 gsm to about 50, 100, or 200 gsm.

The three-dimensional sheet material 10 can be provided with a plurality of first embossments 20 and a plurality of second embossments 30 wherein each of the first embossments 20 and the second embossments 30 extend upwardly and away from the upper surface 14 of the three-dimensional sheet material 10. The first embossments 20 and the second embossments 30 can be provided in any shape deemed suitable for the particular end application. Referring to FIG. 1, the first embossments 20 and the second embossments 30 are each illustrated as having an elongated shape with bulbous ends, otherwise known as being “dog bone” in shape. Referring to FIG. 2, while the upper most portion of the first embossment 20 is illustrated as being flat or substantially flat, it is also possible to have other shaped surfaces as well. For example, the upper most portion of the first embossments 20 and the second embossments 30 can be rounded such as convex.

Each of the first embossments 20 can define a first major axis 60, a first minor axis 62, and a first overall length 64. In various embodiments, the first overall length 64 can be from about 6, 6.5, 7, or 7.5 mm to about 8, 8.5, 9, 9.5 or 10 mm. Each of the first embossments 20 can have an opposing pair of first ends 22 separated from each other by a first central region 24 which has a pair of opposing first sides 26. Each of the first ends 22 has a first end width 66 and the first central region 24 has a first central width 68. The first central width 68 can be less than the first end width 66 of each of the first ends 22. In various embodiments, the first central width 68 can be from about 1, 1.1, 1.2, 1.3, 1.4, or 1.5 mm to about 1.6, 1.7, 1.8, 1.9 or 2 mm. In various embodiments the first end width 66 of each of the first ends 22 can be from about 2, 2.1, 2.2, 2.3, 2.4, or 2.5 mm to about 2.6, 2.7, 2.8, 2.9 or 3 mm.

Each of the second embossments 30 can define a second major axis 70, a second minor axis 72, and a second overall length 74. In various embodiments, the second overall length 74 can be from about 6, 6.5, 7, or 7.5 mm to about 8, 8.5, 9, 9.5 or 10 mm. Each of the second embossments 30 can have an opposing pair of second ends 32 separated from each other by a second central region 34 which has a pair of opposing second sides 36. Each of the second ends 32 has a second end width 76 and the second central region 34 has a second central width 78. The second central width 78 can be less than the second end width 76 of each of the second ends 32. In various embodiments, the second central width 78 can be from about 1, 1.1, 1.2, 1.3, 1.4, or 1.5 mm to about 1.6, 1.7, 1.8, 1.9 or 2 mm. In various embodiments the second end width 76 of each of the second ends 32 can be from about 2, 2.1, 2.2, 2.3, 2.4, or 2.5 mm to about 2.6, 2.7, 2.8, 2.9 or 3 mm.

The first embossments 20 and the second embossments 30 can be incorporated into the three-dimensional sheet material 10 in a manner such that they can form an arrangement with each other. In various embodiments, each of the plurality of first embossments 20 and each of the plurality of second embossments 30 can be incorporated into the three-dimensional sheet material 10 in an arrangement of alternating rows. In various embodiments, the first embossments 20 can be incorporated into the three-dimensional sheet material 10 such that the first major axis 60 of the first embossments 20 is perpendicular, plus or minus 10 degrees, to the first major axis 70 of the second embossments 30. In various embodiments, the distance 80 between a central region of an embossment, such as, second central region 34 of a second embossment 30, and an end of an embossment, such as, first end 22 of a first embossment 20, can be from about 0.2, 0.3, 0.4, or 0.5 mm to about 0.6, 0.7, 0.8, 0.9, or 1 mm.

The three-dimensional sheet material 10 can be provided with a plurality of first apertures 40 and a plurality of second apertures 50. Referring to the FIG. 2, the first apertures 40 are illustrated as extending from the upper surface 14, through the thickness 18 of the three-dimensional sheet material 10, and to the lower surface 16 of the three-dimensional sheet material 10. The second apertures 50 extend downwardly and away from the lower surface 16 of the three-dimensional sheet material 10. It is to be understood that the first apertures 40 can also extend downwardly and away from the lower surface 16 of the three-dimensional sheet material 10 if so desired. The first apertures 40 and the second apertures 50 can be provided in any shape deemed suitable for the particular end application. Referring to FIG. 1, the first apertures 40 and the second apertures 50 are each illustrated as having a circular shape at the upper surface 14 of the three-dimensional sheet material 10. Referring to FIG. 2, the first apertures 40 are illustrated as having sidewalls 42 which extend through the thickness 18 of the three-dimensional sheet material 10 in a straight manner. The second apertures 50 are illustrated as having sidewalls 52 which extend through the thickness 18 of the three-dimensional sheet material 10 at an angle such as to form an aperture in a cone shape. It is to be understood that the first apertures 40 and the second apertures 50 can have any shape as desired for the end application.

Each of the first apertures 40 can define a first width 44 at the upper surface 14 of the three-dimensional sheet material 10 and can have a first depth 46 as defined by the length of the first sidewalls 42. In various embodiments, the first width 44 of the first apertures 40 can be from about 0.4 or 0.5 mm to about 0.6 or 0.7 mm.

Each of the second apertures 50 can define a second width 54 at the upper surface 14 of the three-dimensional sheet material 10 and can have a second depth 56 as defined by the length of the second sidewalls 52. In various embodiments, the second width 54 of the second apertures 50 can be from about 0.8 or 0.9 mm to about 1, 1.1, or 1.2 mm.

In various embodiments, the first width 44 of the first apertures 40 is less than the second width 54 of the second apertures 50. In various embodiments, the second depth 56 of the second apertures 50 is greater than the first depth 46 of the first apertures 40 as measured from the upper surface 14 of the three-dimensional sheet material 10.

The first apertures 40 and the second apertures 50 can be incorporated into the three-dimensional sheet material 10 in a pattern surrounding each of the first embossments 20 and second embossments 30. The first apertures 40 can be positioned such that they are adjacent the first sides 26 of the first central region 24 of the first embossments 20 as well as the second sides 36 of the second central region 34 of the second embossments 30. The second apertures 50 can be positioned such that they are adjacent the first ends 22 of the first embossments 20 as well as the second ends 32 of the second embossments 30. In various embodiments, a second aperture 50 can be positioned a distance 82 from about 0.2 mm to about 0.5 mm from a first end 22 or second end 32 of a first embossment 20 or a second embossment 30, respectively.

In various embodiments, the first ends 22 of the first embossments 20 are separated from the second central region 34 of the second embossments 30 by the first apertures 40. In various embodiments, the second ends 32 of the second embossments 30 are separated from the first central region 24 of the first embossments 20 by the first apertures 40.

As noted herein, in various embodiments, the three-dimensional sheet material 10 can be a single layer of material, such as first layer of material 12. In various embodiments, it may be desirable to incorporate a second layer of material in the three-dimensional sheet material 10. The second layer of material can be any material such as described as appropriate for the first layer of material 12. The second layer of material can have an upper surface, a lower surface and a thickness therebetween. In various embodiments, the upper surface of the second layer of material can be positioned adjacent to the lower surface 16 of the first layer of material 12. In various embodiments the second layer of material can be bonded to the first layer of material 12. As used herein, the term “bonded” refers herein to the joining, adhering, connecting, attaching, or the like, of two elements. Two elements will be considered bonded together when they are joined, adhered, connected, attached, or the like, directly to one another or indirectly to one another, such as when bonded to an intermediate element. The bonding can occur via, for example, adhesive, pressure bonding, thermal bonding, ultrasonic bonding, chemical bonding, stitching, suturing, and/or welding. In various embodiments in which the three-dimensional sheet material 10 has a first layer of material 12 and a second layer of material, the second apertures 50 can extend through the first layer of material 12 and the second layer of material. In various embodiments in which the three-dimensional sheet material 10 has a first layer of material 12 and a second layer of material, each of the first apertures 40 and the second apertures 50 can extend through the first layer of material 12 and the second layer of material.

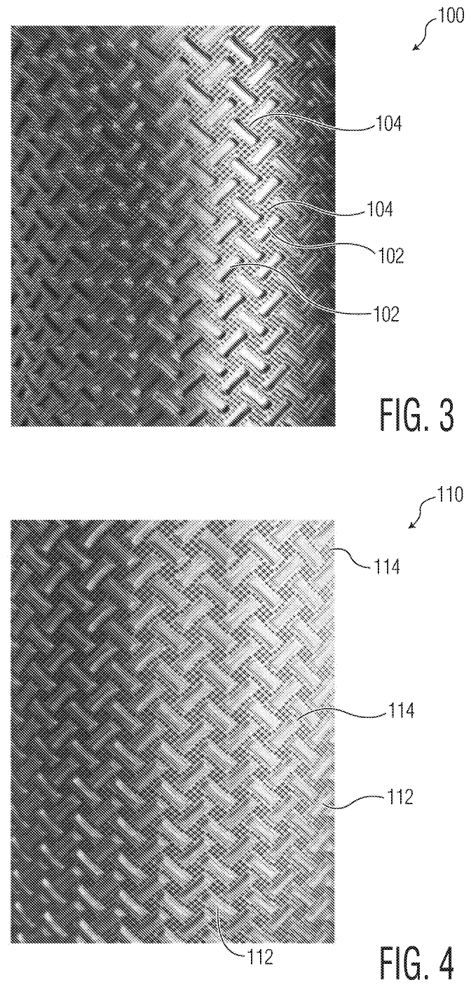

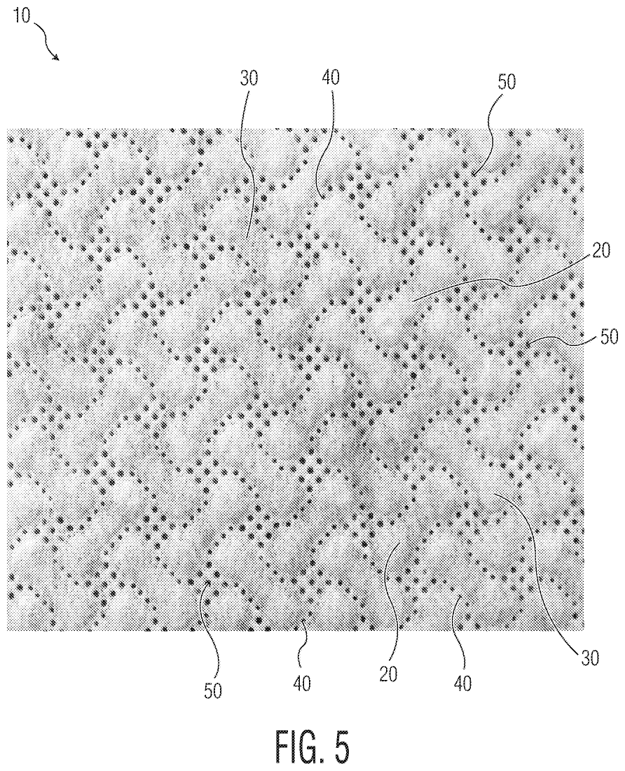

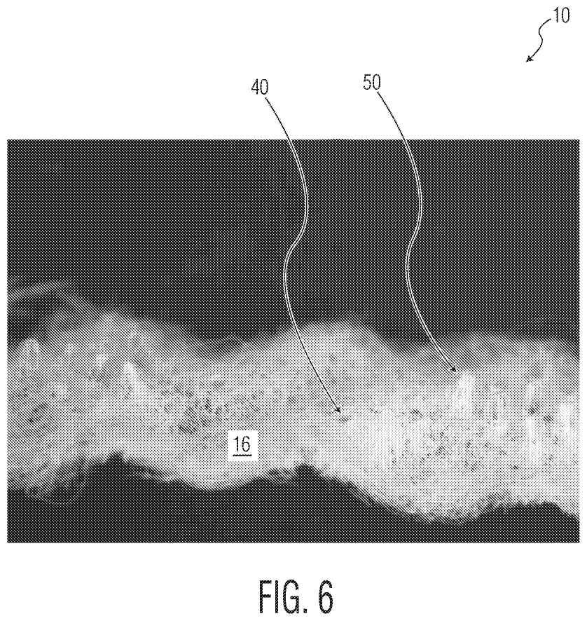

To form the three-dimensional sheet material 10, conventional intermeshing male and female embossing rolls may be used. Referring to FIGS. 3 and 4, FIG. 3 provides an exemplary embodiment of the surface of a female roll 100 and FIG. 4 provides an exemplary embodiment of the surface of a male roll 110. The male roll 110 can be provided with convex embossing units 112 and valleys 114 which can receive perforation pins 104 which are positioned on the surface of and extending outwardly from the surface of the female roll 100. The female roll 100 can also be provided with concave regions 102 which can receive the convex embossing units 112 positioned on the surface of and extending outwardly from the surface of the male roll 110. When the first layer of material 12 is positioned between the female roll 100 and the male roll 110, the three-dimensional shape will be formed resulting in the three-dimensional sheet material 10. FIG. 5 provides an illustration of a top plan view of a three-dimensional sheet material 12 made utilizing and intermeshing a female roll 100 and male roll 110. FIG. 6 provides a perspective view of a three-dimensional sheet material 10. The lower surface 16 is visible to the viewer in the image presented in FIG. 6. As can be seen, the second apertures 50 have a greater depth 56 than the first apertures 40.

The three-dimensional shape set in the first layer of material 12 can be due solely to mechanical pressure. Alternatively, either or both of the female and/or male rolls, 100 and/or 110, may be heated and/or cooled to facilitate the formation and set of the three-dimensional shape. Additionally, ultrasonic equipment using horns and anvils can be used to impart the desired embossing pattern.

If it is desired to coat the upper surface 14 of the three-dimensional sheet material 10 to make it more hydrophobic, a coating roller or other suitable equipment can be placed adjacent the female roll 100 just downstream of the male roller to apply a coating of desired hydrophobic (or hydrophilic, if it is desired to make the material more hydrophilic) material to the upper surface 14 of the three-dimensional sheet material 10.

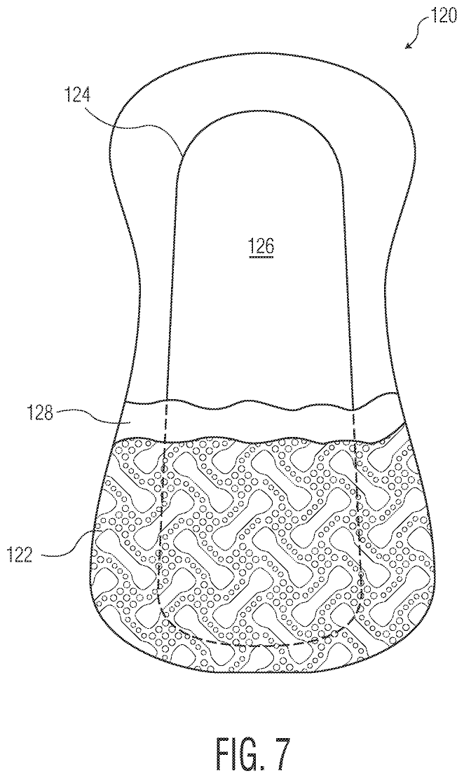

The three-dimensional sheet material 10 can be used in a wide variety of applications including, but not limited to, personal care absorbent articles designed to be worn against or around the body to absorb body exudates. Referring to FIG. 7, there is shown an exemplary absorbent article 120, in this case a sanitary napkin, which employs the three-dimensional sheet material 10 as the liquid pervious topsheet layer 122. The absorbent article 120 also includes a liquid impervious backsheet layer 124 which is typically joined to the topsheet layer 122 either directly or indirectly and an absorbent core 126 is disposed between the topsheet layer 122 and the backsheet layer 124. Optionally, the absorbent article 120 can include other layers such as what is termed a surge layer or transfer layer 128 located between the absorbent core 126 and the topsheet layer 122. While the three-dimensional sheet material 10 is shown as being used for the topsheet layer 122, it may be used for any of the other layers including, but not limited to, the surge layer 128 or the absorbent core 126.

In the interests of brevity and conciseness, any ranges of values set forth in this disclosure contemplate all values within the range and are to be construed as support for claims reciting any sub-ranges having endpoints which are whole number values within the specified range in question. By way of hypothetical example, a disclosure of a range of from 1 to 5 shall be considered to support claims to any of the following ranges: 1 to 5; 1 to 4; 1 to 3; 1 to 2; 2 to 5; 2 to 4; 2 to 3; 3 to 5; 3 to 4; and 4 to 5.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

All documents cited in the Detailed Description are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by references, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

When introducing elements of the present disclosure or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements. Many modifications and variations of the present disclosure can be made without departing from the spirit and scope thereof. Therefore, the exemplary embodiments described above should not be used to limit the scope of the invention.

What is claimed is:

1. A three-dimensional sheet material characterized by comprising a first layer of material comprising:
a. an upper surface and a lower surface defining a thickness therebetween;
b. a plurality of first embossments in which each first embossment defines a first major axis and a first minor axis, and a first overall length;
c. a plurality of second embossments in which each second embossment defines a second major axis and a second minor axis, and a second overall length;
d. a plurality of first apertures in which each first aperture has a first width and a sidewall defining a first depth;
e. a plurality of second apertures in which each second aperture has a second width and a sidewall defining a second depth; wherein
f. the first major axis is perpendicular to the second major axis +/−10 degrees;
g. the first width of at least a portion of the plurality of first apertures is less than the second width of at least a portion of the plurality of the second apertures; and
h. the first depth of at least a portion of the plurality of first apertures is less than the second depth of at least a portion of the plurality of the second apertures.

2. The three-dimensional sheet material of claim 1 wherein
a. the plurality of first embossments extend upwardly and away from the upper surface;
b. the plurality of second embossments extend upwardly and away from the upper surface;
c. the first embossments have opposed first ends separated by a first central region having opposed first sides, each of the first ends having a first end width and the first central region having a first central width, the first central width being less than the first end width of each of the first ends, and
d. the second embossments have opposed second ends separated by a second central region having opposed second sides, each of the second ends having a second end width and the second central region having a second central width, the second central width being less than the second end width of each of the second ends.

3. The three-dimensional sheet material of claim 2 wherein the plurality of second apertures are adjacent the ends of the plurality of first embossments and the plurality of second apertures are also adjacent the second ends of the plurality of second embossments.

4. The three-dimensional sheet material of claim 3 wherein the plurality of first apertures are adjacent the opposed first sides of the first central region of the plurality of first embossments and the plurality of first apertures are also adjacent the opposed second sides of the second central region of the second embossments.

5. The three-dimensional sheet material of claim 4 wherein the second ends of the plurality of second embossments are separated from the first central regions of the plurality of first embossments by the first apertures.

6. The three-dimensional sheet material of claim 5 wherein the first ends of the plurality of first embossments are separated from the second central regions of the plurality of second embossments by the first apertures.

7. The three-dimensional sheet material of claim 6 wherein the second depth of the plurality of second apertures is greater than the first depth of the plurality of first apertures as measured from the upper surface.

8. The three-dimensional sheet material of claim 1 wherein a second layer of material is adjacent the lower surface of the first layer of material, the second layer of material having a top surface and a bottom surface separated by a second thickness.

9. The three-dimensional sheet material of claim 8 wherein the plurality of 1^{st} apertures and the plurality of second apertures extend through the first layer of material and the second layer of material.

10. The three-dimensional sheet material of claim 9 wherein the second layer of material is bonded to the first layer of material.

11. A personal care absorbent article which includes the three-dimensional sheet material of claim 1.

12. A personal care absorbent article which includes the three-dimensional sheet material of claim 8.

13. A personal care absorbent article comprising a topsheet layer and a backsheet layer with an absorbent core located between the topsheet layer and the backsheet layer wherein the topsheet layer includes the three-dimensional sheet material of claim 1.

14. A personal care absorbent article comprising a topsheet layer and a backsheet layer with an absorbent core located between the topsheet layer and the backsheet layer wherein the topsheet layer includes the three-dimensional sheet material of claim 8.

15. The personal care absorbent article of claim 13 wherein the absorbent article is a sanitary napkin.

16. The personal care absorbent article of claim 14 wherein the absorbent article is a sanitary napkin.
